(19) United States
(12) Patent Application Publication
(10) Pub. No.: US 2017/0007329 A1
(43) Pub. Date: Jan. 12, 2017

(54) LEG LENGTH AND OFFSET CALCULATION IN COMPUTER-ASSISTED SURGERY USING RANGEFINDER

(71) Applicant: ORTHOSOFT INC., MONTREAL (CA)

(72) Inventors: DI LI, LASALLE (CA); LOUIS-PHILIPPE AMIOT, HAMPSTEAD (CA); FRANCOIS PARADIS, BOUCHERVILLE (CA); BENOIT PELLETIER, MONTREAL (CA); LAURENCE MOREAU-BELANGER, LAVAL (CA); KARINE DUVAL, MONTREAL (CA)

(21) Appl. No.: 15/203,208

(22) Filed: Jul. 6, 2016

Related U.S. Application Data
(60) Provisional application No. 62/312,509, filed on Mar. 24, 2016.
(60) Provisional application No. 62/188,921, filed on Jul. 6, 2015.

(51) Int. Cl. A61B 34/10; A61B 34/20; A61B 5/00; A61B 5/107

(52) U.S. Cl. CPC A61B 34/10; A61B 5/1075; A61B 34/20; A61B 5/4528; A61B 2034/105; A61B 2034/2055

(57) ABSTRACT

A system for measuring a length variation between body portions in computer-assisted surgery between a preoperative condition and intra- or post-operative condition comprises a a rangefinder configured to measure its distance to at least one reference landmark on at least a first body portion of a patient from a known position relative to a second body portion. A support includes joint(s) allowing one or more rotational degree of freedom of movement of the rangefinder to point to the at least one reference landmark. An inertial sensor unit is connected to the rangefinder to produce orientation data for the rangefinder. A computer-assisted surgery processing unit has a tracking module for tracking the rangefinder in a virtual coordinate system using the orientation data, a coordinate system module for determining coordinates in the virtual coordinate system of the at least one reference landmark using the distance and the orientation data, and a length calculation module for measuring a length between the body portions using the coordinates, the length calculation module calculating and outputting the length variation between the body portions by using said length obtained from a preoperative condition and said length obtained from an intra- or post-operative condition. A method for measuring a length variation between body portions in computer-assisted surgery between a preoperative condition and intra- or post-operative condition is also provided.

### CROSS-REFERENCE TO RELATED APPLICATIONS

The present disclosure claims the priority of U.S. Patent Application Ser. No. 62/188,921, filed on Jul. 6, 2015, and the priority of U.S. Patent Application Ser. No. 62/312,509, filed on Mar. 24, 2016, the content of both applications being incorporated herein by reference.

### TECHNICAL FIELD

The present disclosure relates to a system and method used in Computer-Assisted Surgery (CAS) to provide length measurement, such as for leg length discrepancy and offset measurements, for instance in hip surgeries.

### BACKGROUND OF THE ART

Leg length discrepancy is a change of leg length along the longitudinal axis of the patient, following orthopedic surgery. Offset is the measurement of the translational shift of the leg along a medio-lateral axis of the patient. Both these parameters are relevant during hip surgery, including total hip replacement, acetabular cup implanting, femoral implanting (e.g., head and neck implant, resurfacing). There is a need for systems and methods for determining leg length discrepancy and offset for example.

### SUMMARY

It is aim of the present disclosure to provide novel systems and methods for measuring anatomic lengths in computer-assisted orthopedic surgery.

It is another aim of the present disclosure that the lengths measured are used in leg length discrepancy and offset calculations to assess orthopedic hip surgery.

Therefore, in accordance with an embodiment of the present disclosure, there is a system for measuring a length variation between body portions in computer-assisted surgery between a preoperative condition and intra- or post-operative condition comprising: a rangefinder apparatus comprising a rangefinder configured to measure its distance to at least one reference landmark on at least a first body portion of a patient from a known position relative to a second body portion, a support including at least one joint allowing at least one rotational degree of freedom of movement of the rangefinder to point to the at least one reference landmark, and an inertial sensor unit connected to the rangefinder to produce orientation data for the rangefinder; and a computer-assisted surgery processing unit having a tracking module for tracking the rangefinder in a virtual coordinate system using the orientation data, a coordinate system module for determining coordinates in the virtual coordinate system of the at least one reference landmark using the distance and the orientation data, and a length calculation module for measuring a length between the body portions using the coordinates, the length calculation module calculating and outputting the length variation between the body portions by using said length obtained from a preoperative condition and said length obtained from an intra- or post-operative condition.

Further in accordance with the embodiment, the support in some instances is in some instances configured to fix the rangefinder to the second body portion in the known position.

Still further in accordance with the embodiment, the second body portion in some instances is an elongated bone, and wherein the rangefinder is configured to have its distance measurement direction aligned with a longitudinal axis of the elongated bone.

Still further in accordance with the embodiment, the length calculation module in some instances projects said lengths obtained from the preoperative condition and from the intra- or post-operative condition on a reference plane to calculate the length variation.

Still further in accordance with the embodiment, the patient in some instances is in a supine position and wherein the reference plane is parallel to a plane of the table supporting the patient, the reference plane determined by the coordinate system module using readings from the inertial sensor unit.

Still further in accordance with the embodiment, the reference landmark in some instances is a board reference device including a target board secured to the first body portion.

Still further in accordance with the embodiment, the target board in some instances has a visual scale thereon to indicate a lateral displacement of the second body portion relative to the first body portion between the preoperative condition and the intra- or post-operative condition.

Still further in accordance with the embodiment, the first body portion in some instances is a pelvis, and the second body portion is a femur, the computer-assisted surgery including alterations to a hip joint.

Still further in accordance with the embodiment, the rangefinder apparatus in some instances is configured to be adjacent to the body portions, the tracking module tracking the rangefinder to the known position using the distance and orientation of the rangefinder relative to at least one said reference landmark on the second body portion.

Still further in accordance with the embodiment, the first body portion in some instances is an elongated bone and the first body portion is another bone jointed to the elongated bone, the computer-assisted surgery including alterations to a joint between the elongated bone and the other bone, and wherein the known position of the rangefinder relative to the second body portion comprises two said reference landmarks on the second body portion.

Still further in accordance with the embodiment, the length calculation module in some instances forms a preoperative plane with the coordinates of the reference landmark on the first body portion and of the two said reference landmarks on the second body portion obtained from the preoperative condition, and forms an intra- or post-operative plane with the coordinates of the reference landmark on the first body portion and of the two said reference landmarks on the second body portion obtained from the intra- or post-operative condition, the length calculation module rotating at least one of the planes about an axis passing through the two said reference landmarks on the second body portion to obtain a parallel relation between the planes, to calculate the length variation from the parallel relation.

Still further in accordance with the embodiment, the length calculation module in some instances determines a lateral position variation of the at least one reference landmark on the first body portion in a direction parallel to the axis passing through the two said reference landmarks on the second body portion, to calculate an offset of the first body portion.

Still further in accordance with the embodiment, the first body portion in some instances is a femur, and the second body portion is a pelvis, the computer-assisted surgery including alterations to a hip joint between the femur and the pelvis.

In accordance with another embodiment of the present disclosure, there is provided a method for measuring a length variation between body portions in computer-assisted surgery between a preoperative condition and intra- or post-operative condition comprising: obtaining at least a distance to at least one reference landmark on a first body portion and orientation data from a rangefinder apparatus in a known position relative to a second body portion; tracking the rangefinder apparatus in a virtual coordinate system using the orientation data; determining coordinates in the virtual coordinate system of the at least one reference landmark using the distance and the orientation data; measuring a length between the body portions using the coordinates; and calculating and outputting the length variation between the body portions by using said length obtained from a preoperative condition and said length obtained from an intra- or post-operative condition.

Still further in accordance with the other embodiment, obtaining the distance in some instances comprises obtaining the distance with the rangefinder apparatus being fixed to the second body portion in the known position.

Still further in accordance with the other embodiment, the second body portion in some instances is an elongated bone, and wherein obtaining the distance comprises obtaining the distance with the rangefinder apparatus having its distance measurement direction aligned with a longitudinal axis of the elongated bone.

Still further in accordance with the other embodiment, calculating and outputting the length variation in some instances comprises projecting said lengths obtained from the preoperative condition and from the intra- or post-operative condition on a reference plane.

Still further in accordance with the other embodiment, the patient in some instances is in a supine position and wherein the reference plane is parallel to a plane of the table supporting the patient, the method further comprising in some instances determining the reference plane using the orientation data from the rangefinder apparatus.

Still further in accordance with the other embodiment, the method in some instances is performed with the first body portion being a pelvis, the second body portion being a femur, and the computer-assisted surgery including alterations to a hip joint.

Still further in accordance with the other embodiment, obtaining at least a distance in some instances comprises obtaining a distance and orientation data from the rangefinder apparatus to at least one said reference landmark on the second body portion, and further wherein tracking the rangefinder apparatus to the known position comprises using the distance and orientation data of the rangefinder apparatus relative to the at least one said reference landmark on the second body portion.

Still further in accordance with the other embodiment, the first body portion in some instances is an elongated bone and the first body portion is another bone jointed to the elongated bone, the computer-assisted surgery including alterations to a joint between the elongated bone and the other bone, and wherein tracking the known position of the rangefinder relative to the second body portion comprises using the distance and orientation data of the rangefinder apparatus to two said reference landmarks on the second body portion.

Still further in accordance with the other embodiment, calculating and outputting the length variation in some instances comprises forming a preoperative plane with the coordinates of the reference landmark on the first body portion and of the two said reference landmarks on the second body portion obtained from the preoperative condition, forming an intra- or post-operative plane with the coordinates of the reference landmark on the first body portion and of the two said reference landmarks on the second body portion obtained from the intra- or post-operative condition, and rotating at least one of the planes about an axis passing through the two said reference landmarks on the second body portion to obtain a parallel relation between the planes, the length variation calculated from the parallel relation.

Still further in accordance with the other embodiment, calculating and outputting the length variation in some instances comprises further determining a lateral portion variation of the at least one reference landmark on the first body portion in a direction parallel to the axis passing through the two said reference landmarks on the second body portion, to calculate and output an offset of the first body portion.

Still further in accordance with the other embodiment, the method in some instances is performed with the first body portion being a femur, and the second body portion being a pelvis, the computer-assisted surgery including alterations to a hip joint.

The feature or features of one embodiment may be applied to other embodiments, even though not described or illustrated, unless expressly prohibited by this disclosure or the nature of the embodiments.

Some details associated with the present embodiments are described above and others are described below.

### DESCRIPTION OF THE DRAWINGS

FIG. 1 is a block diagram of a system for measuring leg length and offset in computer-assisted surgery, in accordance with the present disclosure;

FIG. 2 is a perspective view of a rangefinder apparatus of the system of FIG. 1, in accordance with an embodiment;

FIG. 3 is a perspective view of the rangefinder apparatus of FIG. 2, as mounted to a tripod;

FIG. 4 is a perspective view of the rangefinder apparatus of FIG. 2, as mounted to a base for being mounted onto a patient;

FIG. 5 is a perspective view of the rangefinder apparatus of FIG. 4, as mounted onto a patient, for measuring leg length discrepancy;

FIG. 6 is a perspective view of the rangefinder apparatus of FIG. 4, for measuring both leg length discrepancy and offset; and

FIG. 7 is a schematic view of a geometric arrangement of landmarks used in measuring leg length discrepancy and offset with the system of FIG. 1.

### DETAILED DESCRIPTION

In the proposed disclosure, the leg length discrepancy and offset measurements are resolved using three-dimensional trigonometry.

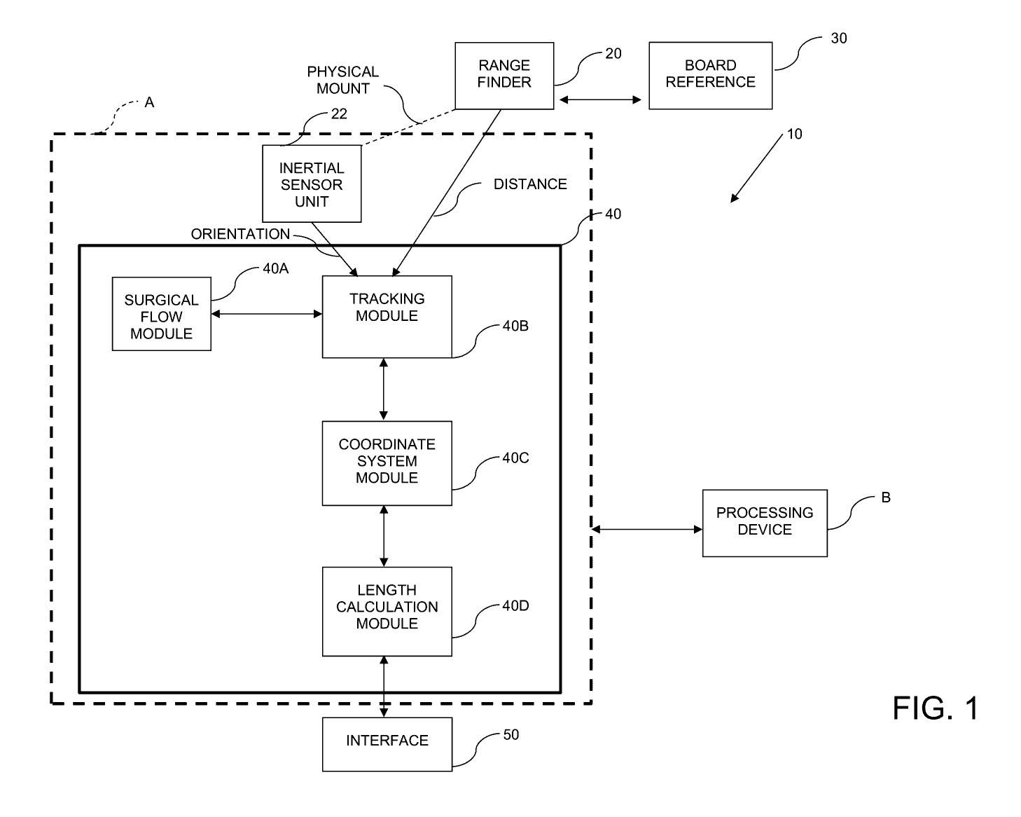

Referring to FIG. 1, a system 10 for measuring leg length in computer-assisted surgery is generally shown as having a rangefinder apparatus 20 (supporting an inertial sensor unit comprising gyroscopes and possibly accelerometers as shown after), an optional board reference device 30, and a computer-assisted surgery (CAS) processor unit 40.

The rangefinder apparatus 20 is of the type having the capacity of outputting distance measurements of a light point it emits on a landmark. The rangefinder apparatus 20 may also be trackable in orientation (pitch, roll and/or yaw) via an inertial sensor unit, optical tracking or equivalent, and possibly in position (X, Y, Z), for instance using optical tracking.

The board reference device 30 may optionally be used to assist in taking the measurements. The board reference device 30 may be a target anchored to the body to serve as an immovable landmark for range measurement.

The CAS processor unit 40 is of the type having a processor and modules adapted to perform calculations to assist an operator during surgery. Accordingly, the CAS processor unit 40 may integrally have or be interfaced to any appropriate interface 50 enabling interaction with the operator, such as a screen (e.g., touchscreen, a display, a monitor, a mouse, keyboard, stylus, etc). While the description provided below refers to the calculation of leg length discrepancy and offset, the CAS processor unit 40 may be used to perform various other functions, with the calculator of leg length discrepancy and offset being one of numerous modules of the system. Alternatively, the system described herein may have the CAS processor unit 40 solely dedicated to the functions described hereinafter of calculating leg length and offset.

Referring to FIGS. 1-3, the rangefinder apparatus 20 comprises a rangefinder 21 that is devised to take accurate distance measurement (e.g., accuracy ±1 mm). For example, the rangefinder 21 may be a laser rangefinder 21, emitting a light point and measuring the distance to the light point with the afore-mentioned accuracy. One possible rangefinder that may be used is the Leica DISTO E7400x rangefinder, provided strictly as an example. As in the illustrated embodiment, the rangefinder apparatus 20 may have a display screen, and may even incorporate the CAS processor unit 40. The rangefinder 21 may alternatively be wired or in wireless communication with the CAS processor unit 40, so as to transmit measurements thereto.

In the methods described hereinafter, three-dimensional (3D) distance and angular measurements are derived from the laser rangefinder 21 and the inertial sensors within the apparatus 20. The rangefinder apparatus 20 may therefore have an inertial sensor unit 22 (e.g. iASSIST POD™ including a tri-axial gyroscope, among other components). The apparatus 20 may be sterile or non-sterile (as detailed hereinafter, it is contemplated to keep the apparatus 20 outside of the sterile zone throughout surgery in the embodiment of FIG. 3), and no preoperative imaging is needed for leg length discrepancy and offset measurements. The laser rangefinder 21 and the inertial sensor unit 22 may be interconnected rigidly, such that there is no relative movement between the two, whereby measurements from the inertial sensor unit 22 are indicative of the orientation of the rangefinder 21. The inertial sensor unit 22 may be designed to be connected in a single possible orientation to the rangefinder 21, such that the orientation of the inertial sensor unit 22 is known relative to the rangefinder 21 to which it is connected when turned on.

According to an embodiment, the laser rangefinder 21 and the inertial sensor unit 22 may be operatively supported by a support 23 that is the headplate of a tripod 24, so as to limit movement of the laser rangefinder 21 to three degrees of freedom (DOFs) of rotation (pitch and yaw, possibly roll as well) only (no translational movement), the laser rangefinder 21 and the inertial sensor unit 22 being rigidly connected to the support 23 to form an integral assembly with its components moving concurrently. As shown in FIG. 3, the support 23 is mounted to the tripod 24 by way of a spherical joint 25. Accordingly, the rangefinder 21 is displaceable in orientation (roll, pitch and yaw), but is fixed in position. Other supporting mechanisms may be used to achieve this, although the tripod 24 is a straightforward and readily available solution. It is also contemplated to provide positional tracking to the rangefinder apparatus 10 (e.g., passive optical tracking), to allow free manipulations of the laser rangefinder 21, instead of limiting same to rotational movements only. However, due to the simplicity of the movement constraint shown with the tripod 24, the disclosure will not elaborate on the positional tracking, although the same geometrical calculations may apply to a system with positional tracking capability.

By being movable, the rangefinder apparatus 20 may point to directly selected landmarks without using any board device, depending on the application. For example, given subcutaneous landmarks may be marked (e.g., marker, sticker) or used due to their visual distinctiveness, throughout leg length calculations, as the rangefinder apparatus 20 with three rotational DOFs can be freely oriented to aim at these landmarks, provided there is a direct line of sight between the patient and the rangefinder 21. The change in orientation will be determined by the inertial sensor unit 22 such that, as the beam illuminates the selected landmarks and a distance is measure, the instant 3D orientation of the rangefinder 21 is recorded. The distance measurement provided by the laser rangefinder 21 is combined to the 3D orientation of the apparatus 20, to perform the trigonometric calculations to obtain leg length discrepancy and/or offset, among other possibilities, as described hereinafter.

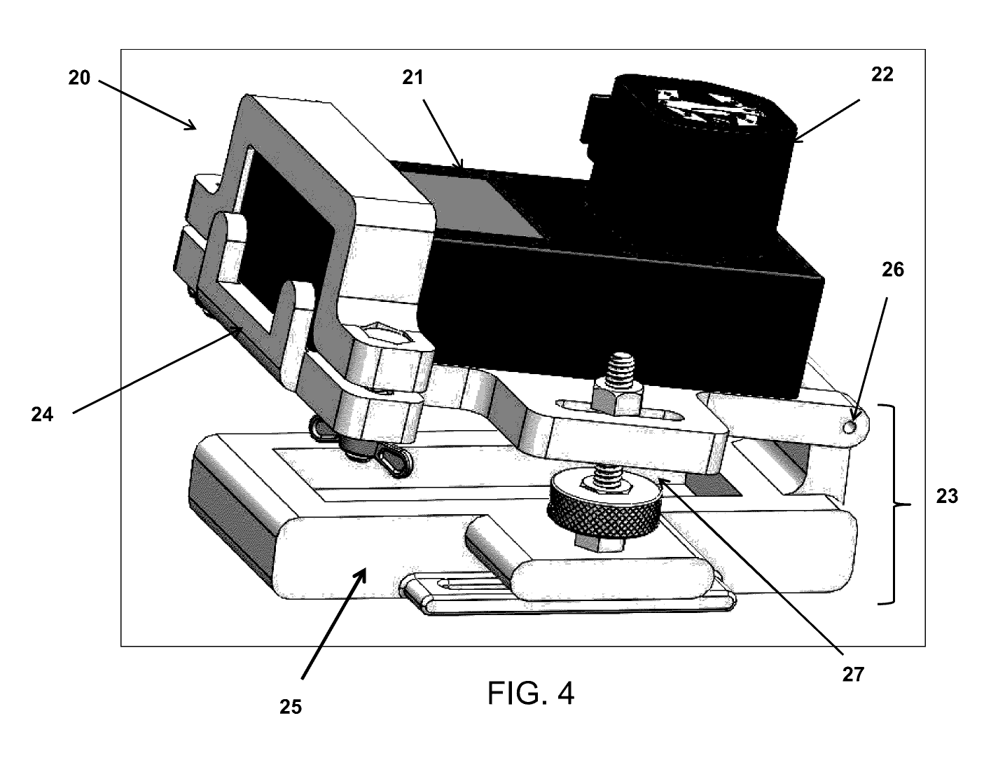

As another arrangement, shown in FIG. 4, the rangefinder apparatus 20 is mounted to a base that is secured to the patient. Its inertial sensor unit 22 may include accelerometers to project the distance and angular measurements into the patient frontal plane, which may be assumed to be the plane of the operating room (OR) table A when the OR table is leveled. The leg length discrepancy and offset measurements may be resolved based on this assumption, as described hereinafter, using trigonometry.

In the embodiment of FIG. 4, the laser rangefinder 21 and the inertial sensor unit 22 may be operatively supported by the support 23 providing a pitch degree of freedom (DOF) of rotation. In FIG. 3, the support 23 is shown as having a sensor interface 24′, upon which are rigidly mounted the laser rangefinder 21 and the inertial sensor unit 22, to form an integral assembly with its components moving concurrently.

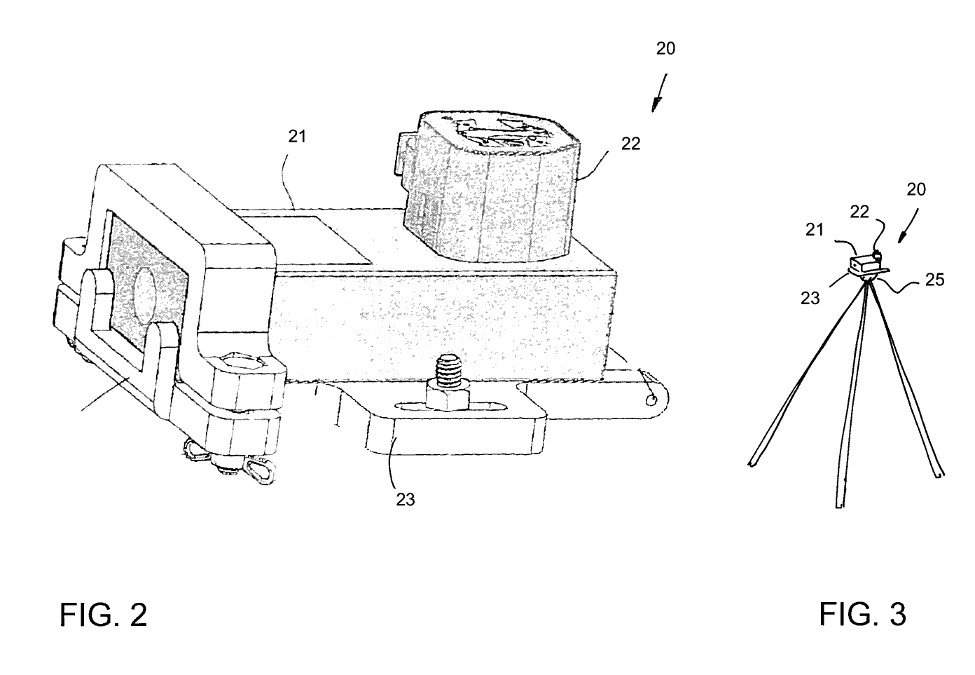

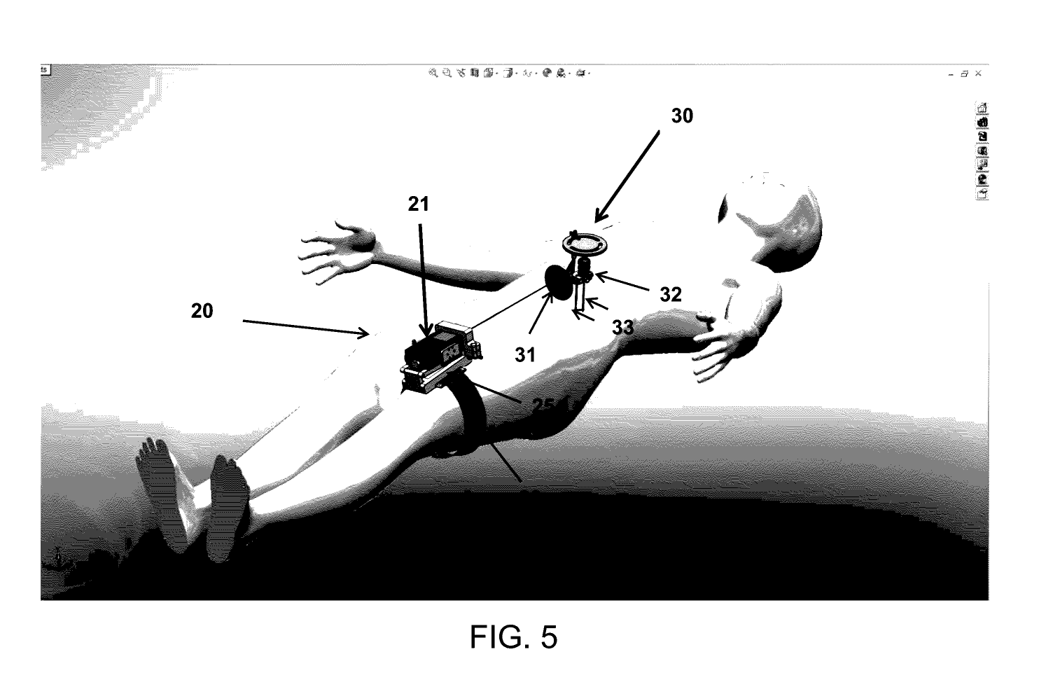

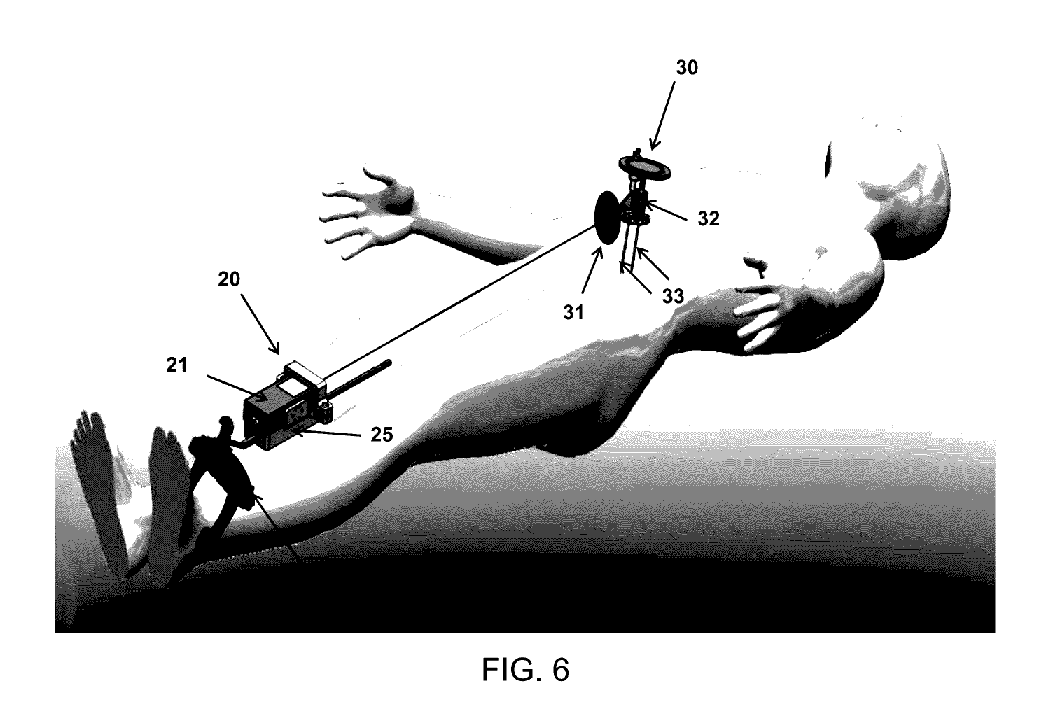

The sensor interface 24′ is mounted to a base 25′, with a lockable rotational degree of freedom (DOF) therebetween. The DOF is provided by hinge joint 26, and by locking feature 27. The locking feature 27 is shown as being a set screw and nut for ease of manual use, but other embodiments are possible. Referring to FIG. 5, a body attachment is a strap 28 rigidly attaching the apparatus 20 to a limb. Referring to FIG. 6, the body attachment may be a caliper 28′ attaching the apparatus 20 to a limb, such as the ankle malleoli. Even though the caliper 28′ secures the apparatus 20 such that the position of the apparatus 20 is fixed, an additional rotational DOF allows angular movements in yaw, such that the orientation of the laser rangefinder 21 may be adjusted to aim the beam of the laser rangefinder 21 on the board reference device 30, or directly on selected landmarks without using board device 30, depending on the application. In similar fashion to the arrangement of FIG. 2, the change in orientation will be determined by the inertial sensor unit 22 such that, once the beam illuminates the target on the board reference device 30 or directly on the selected landmarks without using board device 30, depending on the application, the 3D orientation of the apparatus 20 is recorded. The distance measurement provided by the laser rangefinder 21 is combined to the 3D orientation of the apparatus 20, to perform the trigonometric calculations to obtain leg length discrepancy and/or offset, among other possibilities.

Referring to FIGS. 5 and 6, the board reference device 30 has a target board 31. The device 30 also has a base 32 to be anchored to a body portion. In the illustrated embodiment, the base 32 features a pair of pins 33 that will be fixed to a bone landmark, such as the pelvis. The pins 33 are selected as being a minimally invasive embodiment, but other options are considered, such as non-invasive solutions such as straps, adhesive. According to an embodiment, it is important that the board reference device 30 remains in the exact same position and orientation throughout the measurements (pre- and post-implanting) or that a position and orientation of the board reference device 30 may be replicated with accuracy and precision, if movement of the device 30 is not tracked. It is also contemplated to provide the device 30 with sensors, such as another of the inertial sensor unit 22. As mentioned above, the device 30 is optionally in some applications, but may assist in providing an accurate target and/or increasing visibility.

Referring to FIG. 1, the CAS processing unit 40 may be integral with the inertial sensor unit 22 (or one of them if more than one is present), as schematically and optionally shown as A, or may be as a module of a computer or portable device, among other possibilities, and thus not part of the integral pod A. A user interface(s) 40 outputs the navigation data (e.g., such as leg length discrepancy and offset), whether it be in the form of LED displays, screens, numerical displays, etc. Alternatively, if the CAS processing unit 40 is part of a pod A with the inertial sensor unit 22, the pod A may be connected to a stand-alone processing device B that would include a screen or like monitor, to provide additional display capacity and surface. By way of example, the processing device B is a wireless portable device such as a tablet in a wired or wireless communication with the pod A. The calculations and steps set forth below are not dependent on the physical arrangement between the inertial sensor unit 22 and the processing unit 40.

The processing unit 40 comprises different modules to perform the navigation and output the leg length and offset data. A surgical flow module 40A may be used in conjunction with the user interface 50 or with the processing device B to guide the operator through the steps leading to the navigation. This may entail providing a step-by-step guidance to the operator, such as what landmark distance to record next, and prompting the operator to perform actions, for instance pressing on a “record” interface that is part of the rangefinder apparatus 20 or of the interface 50 or entering data as measured using the rangefinder apparatus 20, for the system 10 to record instant orientations and distances. While this occurs throughout the surgical procedure, the prompting and interactions between the system 10 and the user will not be described in a remainder of the description, as they will implicitly occur. It is contemplated to have the surgical flow module 40A present in the processing device B, with concurrent action between the inertial sensor unit 22 and the processing device B to guide the operator during the measuring procedures detailed below, and with a communication with the operator to record the progress of the procedure.

A tracking module 40B may also be part of the processing unit 30. The tracking module 40B receives readings from the inertial sensor unit 22, converts these readings to useful information, i.e., the navigation data, and records the orientation of the rangefinder 21 as provided by the tracking module 40B, for each landmark distance measurement. As described above, the navigation data may be orientation data in three rotational DOFs for the rangefinder 21, at the instant distance measurements. Accordingly, the tracking module 40B may perform dead reckoning to track the inertial sensor unit 22. Dead reckoning is commonly known and documented and forms part of the common general knowledge, as using inertial sensor readings to continually calculate the orientation and velocity of a body without the need for an external reference, i.e., no signal transmission from outside of the sensor assembly is necessary, the inertial sensor unit 22 is self-contained. An initial orientation and velocity must be provided to the inertial sensor unit 22, e.g., a X-Y-Z coordinate system, after which the orientation is tracked by integrating the angular rates of gyroscope readings at each time step. Since the inertial sensor unit 22 has no need for an external reference, it may be immune to environmental factors such as magnetic fields and operate under a wide range of conditions.

The coordinate system module 40C creates the coordinate system using the orientation data produced by the tracking module 40B, and related distance measurement. The coordinate system is the virtual frame of reference in which the landmarks have coordinates, based on the readings from the rangefinder 21 and the inertial sensor unit 22. For example, the coordinate system module 40C sets a pelvic coordinate system from readings of the rangefinder 21 and the inertial sensor unit 22. The origin of the coordinate system may be arbitrary, for instance using the rangefinder 21 as origin, or superposing one of the landmarks as the origin, for simplicity.

In order to calculate and output the offset and leg length discrepancy, via the user interface 50 or processing device B, the processing unit 40 may have a length calculation module 40D. The length calculation module 40D uses the coordinates of the landmarks in the coordinate system created by the coordinate system module 40C, to calculate the leg length discrepancy and/or the offset, using 3D trigonometry. The length calculation module 40D may therefore output the leg length discrepancy and offset.

Now that the system 10 has been described, a method for measuring leg length and offset is set forth. The patient may be positioned in any appropriate position, including supine decubitus and lateral decubitus, provided there is a line of sight between the rangefinder 21 and the landmarks discussed hereinafter. The rangefinder apparatus 20 is positioned in such a way that there is an unobstructed line of sight between the rangefinder 21 and the desired landmarks on the patient. For example, the rangefinder apparatus 20 with tripod 24 of FIG. 3 may be positioned adjacent to the patient. The rangefinder apparatus 20 connected to the patient by the caliper 28′ in FIG. 6 may also be used with this approach.

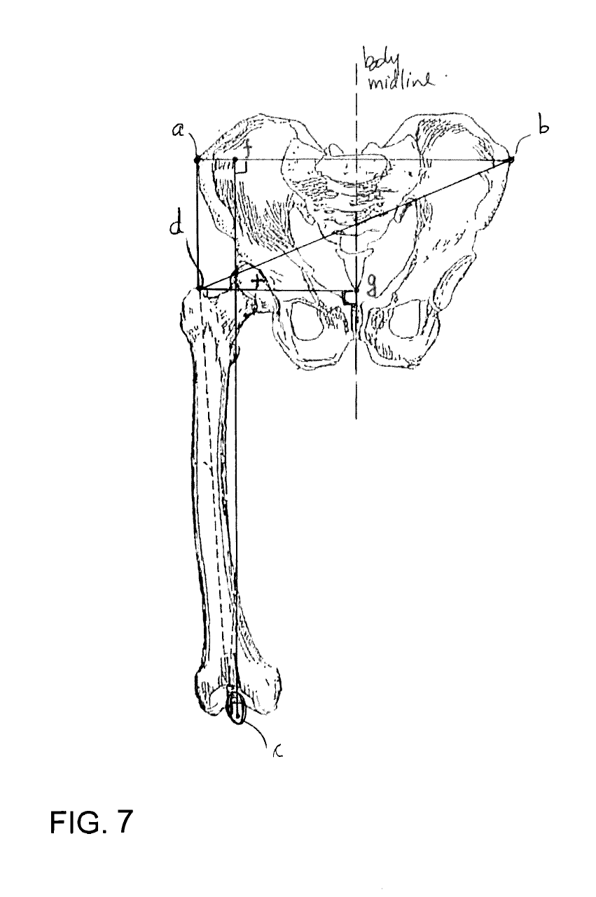

In the embodiment involving the rangefinder apparatus 20 of FIG. 3 or of FIG. 6, laser distance measurements are performed on chosen landmarks, for example before resection, and after implanting, also referred to as preoperatively and postoperatively. The landmarks may be any appropriate landmarks representative of leg length and offset variations relative to a remainder of the body. According to an embodiment, as shown in FIG. 7, two reference landmarks are taken on the pelvis and are used as a reference for distance variations. For clarity, the pelvis and femur are shown without soft tissue. However, the reference landmarks are generally covered with soft tissue, with the exception of one femoral landmark. For example, the reference landmarks on the pelvis may be \textcircled{1} the left ASIS (antero-superior iliac spine), and \textcircled{2} the right ASIS. The ASIS are visually distinct even when covered by skin. Other pelvic landmarks may be used, for instance using added markers onto the patient's skin. For example, it may be difficult to place patients with high body-mass indexes in lateral decubitus as the ASIS may not be visible with the rangefinder 21. In such cases, other landmarks on the pelvis may be used.

Two femoral landmarks may be used, such as \textcircled{3} patella for leg length calculation due to its visual distinctiveness when covered by skin, and the exposed greater trochanter (i.e., not covered by soft tissue), for offset calculation. Other landmarks could be used as well, such as a random point near the greater trochanter for offset, and a random point on the tibia or the femur for leg length discrepancy, provided the landmarks are the same through the procedure.

In the embodiment of FIG. 3 or of FIG. 6, the instant distance measurements from the laser rangefinder 21 and corresponding orientation data from the inertial sensor unit 22 are recorded by the tracking module 40B of the CAS processing unit 40, for these four landmarks, pre- and postoperatively. The leg length and offset can then be obtained by mathematical calculations in the CAS processing unit 10 based on resolving 3D trigonometry, using the combined actions of the coordinate system module 40C and length calculation module 40D.

Referring to FIG. 7, the landmarks (2 ASISs, patella, the greater trochanter) are projected on a reference plane where the leg length and offset calculations take place. The projected landmarks are shown, where:

a, b=two ASISs projected on the reference plane;
c=patella projected on the reference plane;
d=greater trochanter projected on the reference plane;
Leg length (cf)=the distance between the lines connecting two ASISs (ab) and patella (c);
Leg offset (dg)=the distance between the body midline (i.e., midpoint between the two ASISs) and greater trochanter (d);
Leg length and offset discrepancy (Δcf, Δdg)=the difference between the preoperative and postoperative leg length and offset measurements. When the three sides of the triangles (abc, abd) are known, the triangles are resolved; therefore, leg length (cf) and offset (dg) are calculated by the CAS processing unit 40.

The CAS processor unit 40 guides an operator in taking these landmark measurements, for instance using the surgical flow module 40A. The CAS processor unit 40 may then record sets of orientation and distance for each landmark using the tracking module 40B. The coordinate system module 40C converts the orientation and distance measurements to coordinates, using the orientation data from the tracking module 40B and the corresponding distance measurements from the rangefinder 21. The length calculation module 40D may then calculate Act, Adg once all landmark coordinates are obtained pre- and postoperatively.

According to an embodiment, the length calculation module 40D uses the line a,b as a reference axis, as it is the same preoperatively and postoperatively. Therefore, when calculating leg length measurement, the sides a,b (i.e., the reference axis) of the preoperative triangle abc and of its counterpart postoperative triangle abc are superposed. Preoperative and intra- or post-operative planes, respectively including preoperative and intra- or post-operative triangles abc are then relatively rotated about the reference axis (sides a,b) for the triangles abc to be in a same plane, referred to as the reference plane (it is noted that the reference plane may be coincident with the preoperative triangle abd). When the triangles abc are in the reference plane, leg length cf may be calculated by the length calculation module 40D.

Likewise, according to the embodiment, the line a,b is used as a reference axis in the offset calculation as well. Therefore, when calculating offset, the sides a,b (i.e., the reference axis) of the preoperative triangle abd and of its counterpart postoperative triangle abd are superposed. The two triangles abd are then rotated about the reference axis (sides a,b) for the triangles abd to be in the same reference. When the triangles abd are in the reference plane, the offset dg may be calculated by the length calculation module 40D. The reference plane for the offset may be different or the same as the reference plane for the leg length calculation.

The method described above does not require that the leg be repositioned in the same plane as it was prior to resection, as the triangles abc and abd are virtually aligned in the reference plane. It is however desired that the general longitudinal leg alignment be preserved, for instance using visual or mechanical assistance to do so, or by aligning the operated leg with the non-operated leg.

In the case of the embodiment of FIGS. 4, 5 and 6, the accelerometers in the inertial sensor unit 22 may be used to assist in transforming the readings to “reference plane” values, with the calculation approach of FIG. 7 being used once more. For example, the landmarks (2 ASISs, patella, the greater trochanter) are projected on the frontal plane where the leg length and offset calculations take place. This is an optional feature, but may simplify the calculations. The frontal plane is obtained by using the gravity acquired by the accelerometer in the inertial sensor unit 22, based on the assumption that the OR table plane represents the patient frontal plane when the patient is in supine decubitus.

Referring to FIG. 5, the system 10 is used to measure the leg length discrepancy. The apparatus 20 is rigidly attached to the femur by the strap 28. The distance measurement will be taken from the distal femur to the pelvis using the laser rangefinder 21. The strapped-down fixation 28 ensures the apparatus 20 is securely fastened on the distal femur with no or minimum movement when the leg undergoes movement during resection and implanting. Moreover, this strapped-down fixation 28 may ensure that the orientation of the laser rangefinder 21 follows the longitudinal axis of the patient, instead of allowing the yaw orientation adjustment of the laser rangefinder 21 relative to the longitudinal axis. As shown, the apparatus 20 has fewer DOFs of movement and cannot be fully adjusted in orientation. The board reference device 30, upon which the laser beam is to be targeted, is pinned to the iliac crest. The orientation and position of the target board 31 could be adjusted in this embodiment to make up for the lack of adjustments provided at the apparatus 20, relative to its base 32; therefore, in this embodiment, the target board 31 can be aligned with the laser beam along the patient longitudinal axis. When the OR table is leveled, it is again assumed that the OR table plane represents the patient frontal plane when the patient is in supine. The distance from the laser rangefinder 21 to the target board 31 and the inclination of the laser rangefinder 21 with respect to horizontal can be obtained from its readings. Then, the distance projected on the horizontal plane can be calculated based on simple trigonometry. As the distance measurement direction (e.g., the laser beam) is aligned with the patient longitudinal axis, the projected distance measurement in the patient frontal plane represents the leg length. Taking the leg length measurement prior to and after the surgery provides leg length discrepancy. The target board 31 may have a scale thereon, for the lateral movement to be noted before and after surgery to obtain a visual reading of offset.

Another embodiment may include positioning the rangefinder apparatus 20 at an output end of a robotic arm. In such a case, the rangefinder apparatus 20 may be with or without an inertial sensor unit 22. The CAS processing unit 40 may use orientation data obtained from the control of the robotic arm instead of data from an inertial sensor unit. For example, the robotic arm may be a serial arm with encoders at joints to determine the position and orientation of the rangefinder apparatus 20.

Therefore, the system 10 may be used to execute a method for measuring a length variation between body portions in computer-assisted surgery, between a preoperative condition and intra- or post-operative condition. According to the method, a distance to at least one reference landmark on a first bone and orientation data from the rangefinder apparatus 20 is obtained in a known position relative to a second bone. The rangefinder apparatus 20 is tracked in a virtual coordinate system using the orientation data. Coordinates in the virtual coordinate system of the at least one reference landmark are determined using the distance and the orientation data. A length is measured between the bones using the coordinates. The length variation between the body portions is calculated and output by using the length obtained from a preoperative condition and said length obtained from an intra- or post-operative condition. The distance may be obtained with the rangefinder apparatus 20 being fixed to the second body portion in the known position.

1. A system for measuring a length variation between body portions in computer-assisted surgery between a preoperative condition and intra- or post-operative condition comprising:
a rangefinder apparatus comprising a rangefinder configured to measure its distance to at least one reference landmark on at least a first body portion of a patient from a known position relative to a second body portion, a support including at least one joint allowing at least one rotational degree of freedom of movement of the rangefinder to point to the at least one reference landmark, and an inertial sensor unit connected to the rangefinder to produce orientation data for the rangefinder; and
a computer-assisted surgery processing unit having a tracking module for tracking the rangefinder in a virtual coordinate system using the orientation data, a coordinate system module for determining coordinates in the virtual coordinate system of the at least one reference landmark using the distance and the orientation data, and a length calculation module for measuring a length between the body portions using the coordinates, the length calculation module calculating and outputting the length variation between the body portions by using said length obtained from a preoperative condition and said length obtained from an intra- or post-operative condition.

2. The system according to claim 1, wherein the support is configured to fix the rangefinder to the second body portion in the known position.

3. The system according to claim 2, wherein the second body portion is an elongated bone, and wherein the rangefinder is configured to have its distance measurement direction aligned with a longitudinal axis of the elongated bone.

4. The system according to claim 1, wherein the length calculation module projects said lengths obtained from the preoperative condition and from the intra- or post-operative condition on a reference plane to calculate the length variation.

5. The system according to claim 4, wherein the patient is in a supine position and wherein the reference plane is parallel to a plane of the table supporting the patient, the reference plane determined by the coordinate system module using readings from the inertial sensor unit.

6. The system according to claim 2, wherein the reference landmark is a board reference device including a target board secured to the first body portion.

7. The system according to claim 6, wherein the target board has a visual scale thereon to indicate a lateral displacement of the second body portion relative to the first body portion between the preoperative condition and the intra- or post-operative condition.

8. The system according to claim 2, wherein the first body portion is a pelvis, and the second body portion is a femur, the computer-assisted surgery including alterations to a hip joint.

9. The system according to claim 1, wherein the rangefinder apparatus is configured to be adjacent to the body portions, the tracking module tracking the rangefinder to the known position using the distance and orientation of the rangefinder relative to at least one said reference landmark on the second body portion.

10. The system according to claim 9, wherein the first body portion is an elongated bone and the first body portion is another bone jointed to the elongated bone, the computer-assisted surgery including alterations to a joint between the elongated bone and the other bone, and wherein the known position of the rangefinder relative to the second body portion comprises two said reference landmarks on the second body portion.

11. The system according to claim 10, wherein the length calculation module forms a preoperative plane with the coordinates of the reference landmark on the first body portion and of the two said reference landmarks on the second body portion obtained from the preoperative condition, and forms an intra- or post-operative plane with the coordinates of the reference landmark on the first body portion and of the two said reference landmarks on the second body portion obtained from the intra- or post-operative condition, the length calculation module rotating at least one of the planes about an axis passing through the two said reference landmarks on the second body portion to obtain a parallel relation between the planes, to calculate the length variation from the parallel relation.

12. The system according to claim 11, wherein the length calculation module determines a lateral position variation of the at least one reference landmark on the first body portion in a direction parallel to the axis passing through the two said reference landmarks on the second body portion, to calculate an offset of the first body portion.

13. The system according to claim 9, the first body portion is a femur, and the second body portion is a pelvis, the computer-assisted surgery including alterations to a hip joint between the femur and the pelvis.

14. A method for measuring a length variation between body portions in computer-assisted surgery between a preoperative condition and intra- or post-operative condition comprising:
obtaining at least a distance to at least one reference landmark on a first body portion and orientation data from a rangefinder apparatus in a known position relative to a second body portion;
tracking the rangefinder apparatus in a virtual coordinate system using the orientation data;
determining coordinates in the virtual coordinate system of the at least one reference landmark using the distance and the orientation data;
measuring a length between the body portions using the coordinates; and
calculating and outputting the length variation between the body portions by using said length obtained from a preoperative condition and said length obtained from an intra- or post-operative condition.

15. The method according to claim 14, wherein obtaining the distance comprises obtaining the distance with the rangefinder apparatus being fixed to the second body portion in the known position.

16. The method according to claim 15, wherein the second body portion is an elongated bone, and wherein obtaining the distance comprises obtaining the distance with the rangefinder apparatus having its distance measurement direction aligned with a longitudinal axis of the elongated bone.

17. The method according to claim 15, wherein calculating and outputting the length variation comprises projecting said lengths obtained from the preoperative condition and from the intra- or post-operative condition on a reference plane.

18. The method according to claim 17, wherein the patient is in a supine position and wherein the reference plane is parallel to a plane of the table supporting the patient, the method further comprising determining the reference plane using the orientation data from the rangefinder apparatus.

19. The method according to claim 14, wherein obtaining at least a distance comprises obtaining a distance and orientation data from the rangefinder apparatus to at least one said reference landmark on the second body portion, and further wherein tracking the rangefinder apparatus to the known position comprises using the distance and orientation data of the rangefinder apparatus relative to the at least one said reference landmark on the second body portion.

20. The method according to claim 19, wherein the first body portion is an elongated bone and the first body portion is another bone jointed to the elongated bone, the computer-assisted surgery including alterations to a joint between the elongated bone and the other bone, and wherein tracking the known position of the rangefinder relative to the second body portion comprises using the distance and orientation data of the rangefinder apparatus to two said reference landmarks on the second body portion.
